(19) United States
(12) Patent Application Publication
(10) Pub. No.: US 2010/0203572 A1
(43) Pub. Date: Aug. 12, 2010

(54) METHOD FOR CARRYING OUT AND EVALUATING MIX & MEASURE ASSAYS FOR THE MEASUREMENT OF REACTION KINETICS, CONCENTRATIONS AND AFFINITIES OF ANALYTES IN MULTIPLEX FORMAT

(75) Inventors: Werner Lehmann, Bronkow (DE); Alexander Böhm, Rabenau (DE); Kai Grossmann, Dresden (DE); Rico Hiemann, Thiendorf Ot Sacka (DE); Jörg Nitschke, Schipkau Ot Klettwitz (DE); Stefan Rödiger, Gera (DE)

Correspondence Address: LONDA, BRUCE S.;NORRIS MCLAUGHLIN & MARCUS, PA, 875 THIRD AVE, 8TH FLOOR, NEW YORK, NY, 10022, US

(73) Assignees: ATTOMOL GMBH MOLEKULARE DIAGNOSTIKA, BRONKOW (DE); FACHHOCHSCHULE LAUSITZ, SENFTENBERG (DE)

(21) Appl. No.: 12/664,246

(22) PCT Filed: Jun. 13, 2008
(86) PCT No.: PCT/EP08/57515

§ 371 Date: Apr. 8, 2010

(30) Foreign Application Priority Data
Jun. 13, 2007 (DE) 10 2007 027 779.4
Jun. 29, 2007 (DE) 10 2007 031 137.2
Feb. 8, 2008 (DE) 10 2008 008 485.9

(51) Int. Cl. C12Q 1/02; G01N 21/76; C12Q 1/00

(52) U.S. Cl. 435/29; 436/172; 435/40.5

(57) ABSTRACT

The invention relates to a method comprising the following steps: a) use of a support which has at least two different microparticle populations immobilized thereon; b) measuring the fluorescence of the support from step a) with an optical resolution 1, said resolution 1 permitting differentiation of microparticle singlets, doublets, triplets, multiplets and monolayers and determination of the localized position of individual immobilized microparticles; c) contacting the support from step a) with the sample to be analyzed; d) performing at least one additional measurement of the fluorescence of the support during or after contacting in accordance with step c) with a resolution 2; e) assigning the fluorescence values measured with resolution 2 to the individual microparticle singlets, doublets, triplets, multiplets and monolayers locally identified on the support in accordance with step b) and assigned to a particular acceptor molecule population; f) determining the change in fluorescence. The method is used to determine reaction kinetics, concentrations and affinities of analytes in samples.

The invention generally relates to the fields of nucleic acid amplification, enzymology and immunology. More specifically, the invention relates to a method for the real-time measurement of reaction kinetics in a “mix & measure” test format wherein a number of parameters are simultaneously (multiplex) recorded and evaluated. The invention is also directed to a multi-color fluorescence measuring system with thermal control, a bead array as well as a kit for the detection of target molecules in said array, preferably for liquid-phase PCR, solid-phase PCR and multiplex PCR.

Methods for the determination of analytes in a sample have been conducted in real time in the form of mix & measure assays so as to save cost and time when performing the determination or improve the accuracy and robustness of the tests. Such tests have been used e.g. for pharmaceutical high-throughput screening or nucleic acid analytics in medical diagnostics.

In general, it is very important to control the reaction temperature, i.e. maintain the temperature at a constant level, build up a gradient, or thermally control rapid thermocycles identically or with different profiles while the measuring parameters are continuously recorded and processed. Frequently, fluorescence signals are detected because they allow combination with a mix & measure assay format in a variety of ways. Changes in fluorescence intensities, fluorescence lifetime or fluorescence polarization, for example, can be indicative of changing concentrations of analytes in samples or intermolecular interactions and can be measured free of contact, i.e. in various test formats in sealed reaction vessels. Mix & measure test formats are understood to involve analytic procedures where all reacting components remain in a single measuring compartment until the signals are detected.

For example, methods for the detection and quantitative determination of nucleic acids, particularly the polymerase chain reaction (PCR), are known to require all the above-mentioned preconditions to be performed optimally. The PCR can be used to obtain both qualitative and quantitative information. Quantitative determination of PCR products can be done in various ways. More recent detection methods allow measurement of in vitro syntheses of PCR products in real time, i.e. in a homogeneous manner and immediately in the liquid phase of the respective PCR vessel used. This so-called real-time PCR is a particularly sensitive method. It involves monitoring the formation of PCR products in each PCR cycle. As a rule, measurement of amplification is carried out in thermocyclers including additional agents for the measurement of fluorescence signals during the amplification reaction. The amplification products are detected e.g. via fluorescence-labeled hybridization samples showing fluorescence only when bound to the target nucleic acid, or via fluorescent dyes binding double-stranded DNA. A defined signal threshold value is established for all analyzed reactions, and the number of cycles (Cp) required to reach the threshold value is determined for both the target nucleic acid and the reference nucleic acids. Thus, on the basis of the Cp values obtained for both the target nucleic acid and the reference nucleic acid, it is possible to determine either absolute or relative numbers of copies of the target molecule.

A more recent method of detecting specific nucleic acid molecules utilizes so-called “molecular beacons” (Tyagi et al., U.S. Pat. No. 6,150,097 A). Molecular beacons are dye-labeled oligonucleotides having a stem-loop structure. Each of the two free ends of the stem sections (3′ and 5′ ends) has a fluorophore coupled thereto, one of the latter acting as a reporter dye and the other as a quencher dye which, given sufficient spatial proximity, quenches the fluorescence of the reporter dye via Förster resonance energy transfer (FRET). The sequences of the stem sections at both ends of the molecular beacon are selected in such a way that the stem sections upon folding of the molecular beacon hybridize solely to each other, but not to other sections of the oligonucleotide. In the state of hybridized stem sections the distance between the reporter and quencher dyes is sufficiently small, so that the fluorescence dye does not fluoresce even in the presence of suitable excitation with light. The loop section has a sequence which is complementary to the sequence of a target sequence. If the molecular beacons and the nucleic acid molecules having the target sequence are present in a solution, the loop sections and the target sequence sections can undergo hybridization, as a result of which the molecular beacon unfolds to break the hybridization of the two stem sections. Such unfolding results in a greater spatial distance between the reporter dye and the quencher dye, and the reporter dye is excited to fluoresce. During continuous observation of the fluorescence intensity, an increase of the intensity can be determined when the molecular beacons come close to the target sequences and hybridize thereto. In this way, the nucleic acid molecules can be detected quantitatively. The molecular beacons involve the substantial drawback of complex and costly synthesis because these probes must be labeled with both fluorescent dye and quencher dye. Quencher-fluorescent dye systems have been used in comparable probe systems such as Amplifluor primers, Scorpions and TaqMan probes.

Another well-known system is the LightCycler system wherein the fluorescent dyes are distributed on two different oligonucleotides. One fluorescent dye (e.g. fluorescein) is located at the 3′ end of the first oligonucleotide, and another fluorescent dye (e.g. LC Red 640) is located at the 5′ end of the oligonucleotide attaching downstream. Fluorescein is excited by an LED as light source and emits light which excites the second fluorescent molecule via FRET. The light emitted by the second dye is measured through a fluorescence filter using a detector. Excitation of the second dye can only take place when the two dye molecules are in spatial proximity (a distance of 1 to 5 nucleotides) as a result of attachment of the two oligonucleotides to their complementary strand. The principle of this system is based on the secondary excitation of a second fluorescent dye, which is not possible until the PCR product has been generated.

The above-mentioned systems can be employed for multiplex uses but, when detecting different target sequences in parallel, are disadvantageous in that the reaction batch becomes relatively expensive due to the increasing number of different detection probes, and that the increasing number of probes might impair the amplification reaction. Furthermore, the number of known FRET pairs is not sufficient to achieve a high multiplex level.

Technical implementation of nucleic acid assays for routine investigations involves the following important issues: homogeneous assay, multiplex procedure as well as microarrays (also referred to as gene chips or biochips). In a multiplex PCR using the above primer/probe system or kit, the method described above allows combination of a number of primer sets which, owing to the different sequences, permit detection of a plurality of target sequences, but usually no more than five, in a single reaction batch. Qualitative measurements are performed in such a way that a previously defined number of amplification cycles is followed by checking whether the concentration of the amplified nucleic acid molecules has exceeded a specific threshold value. For quantification, this concentration is recorded after each cycle, and the number of cycles to reach a specific threshold value is determined. This number is a measure of the concentration of the searched-for nucleic acid in a sample.

The real-time PCR has become widespread for two properties that rarely go together well in laboratory daily routine: it is both rapid and precise. There is hardly any real-time cycler that would take longer than two hours to multiply and quantify minute amounts of DNA. Real-time cyclers whose cycler blocks can be equipped with appropriate microtiter plates also work in high-throughput mode with up to 384 simultaneous PCRs.

A large number of real-time cyclers are known. They allow detection of preferably up to five fluorescent dyes for multiplex uses at the same time, have heating blocks preferably in packs of four for 96- or 384-well microtiter plates, show rapid heating/cooling rates allowing e.g. 30 PCR cycles within 30 minutes, have quantification software, melting point analysis and many other things. The system and method according to the invention are also suitable for high-throughput laboratories where enormous numbers of samples must be processed, thereby allowing up to 5000 analyses per day with suitable automated real-time PCR robots. Where real-time quantification is not required and the only point of interest is end-point PCR, up to 30,000 samples in less than 3 hours can be possible with appropriate PCR robots.

One alternative of increasing the multiplex level is hybridization of the PCR products to a DNA array bearing on its surface specific capture probes at spatially distinct points. By detecting a hybridization reaction it is possible to detect amplified DNA molecules or characterize the sequence thereof.

The principle of an array experiment is simultaneous hybridization of all gene samples present on the array with a nucleic acid sample. One crucial point is that, in ideal circumstances, the cDNA obtained by reverse transcription of RNA from cells or tissue comprises all genes specifically expressed therein. Parallel hybridization of a nucleic acid sample with a large number of complementary gene samples on a DNA array results in a characteristic hybridization pattern with corresponding hybridization intensity. This reveals the decisive advantage of this technology over other methods of investigating gene expression. While conventional methods are restricted to the investigation of single genes, a DNA array provides a more comprehensive gene expression profile of the cell or tissue under investigation. Of particular interest is the resulting option of investigating the interaction of different genes. Detection of differences in gene expression is possible by simultaneous hybridization of normal/immortalized or fetal/adult cells. The basic element of chips currently used is preferably a glass or plastic support, such as those used in microscopy. The probes (preferably on microparticles) are placed on such a support using e.g. a robot or lithography. The method must proceed with high precision and cleanliness. Billionths of a liter (normally 0.6 nl) of oligonucleotide solution are used for each spot. Each run is followed by self-cleaning of the needles (rinse, ultrasound) so as to avoid spreading of impurities. The oligonucleotides have a reactive group such as an amino group (NH₂) at one end thereof, which stably binds to the support or to the microparticle via chemical reaction. The spots have a diameter of preferably 100 μm (0.1 mm). Owing to the small quantity and the small distance between individual points (preferably 0.3 mm) up to 30,000 spots can be accommodated on a single glass support. The spots serve as future docking sites for sample DNA. Unused areas of the support must be blocked in a final step. This is done by treatment with a special blocking solution which may already be included in the ready-to-use kit. The specific target genes are multiplied by means of PCR, subjected to fluorescence coupling, placed on the support and incubated for hybridization. Unbound DNA is optionally removed by washing several times. A special scanner irradiates the chips with light of a suitable wavelength, and the fluorescent dyes are excited to fluorescence. The result obtained therefrom is subsequently evaluated, with luminous spots indicating a positive result.

Apart from site-encoded arrays where biomolecules are e.g. printed in fixed arrangement on planar supports, bead arrays utilize an encoded support provided with particles. In the multiplex approach, particles with different coding are coated with different biomolecules. The particles themselves are subsequently immobilized on a planar support in random distribution. Binding of molecules from the sample to be analyzed to the respective particles and labeling thereof (ligand labeling) is followed by detecting the coding and the ligands and there-from assigning ligand binding to the immobilized biomolecules.

Assessment of the arrays is a relatively time-consuming process. Especially in kinetic measurements using a number of measurement points in a single measurement, unacceptable time delays arise because imaging and processing of the images is highly complex. Such test systems are usually miniaturized to reduce material expenses originating from multiplexing, so that sufficiently high optical resolution is required which in turn gives rise to large amounts of data. Another drawback of arrays is their lack of reproducibility in manufacturing and the associated fluctuation of measured values obtained through the use of arrays. Compared to planar assays, bead arrays have advantages, but assessment is slowed down due to the coding of the particulate support because a number of images (usually 3-4) per object must be recorded and processed. In addition, the arrays up to now have rarely been operated as mix & measure assays, implying complex handling and risk of contamination in the event of nucleic acid arrays. More-over, quantification of the results is therefore only possible with great difficulties.

The invention was therefore based on the object of providing an improved assay system for multiplex analyses, which system complies with at least one of the following requirements:
robust array platform
mix & measure detection method for biomolecules
multiplex-capable, rapidly temperature-controllable measuring system
ultra-rapid data analysis adjusted to the hardware of the measuring system, which permits reduction of data per object and point of measurement and limits the amount of image processing to a minimum.

In a preferred fashion the invention serves to solve analytical problems which require recording a number of measurement points per measuring series, e.g. in kinetic measurements or thermocycling methods, or in those cases where high sample throughput must be ensured, e.g. in screenings of pharmaceutical active substances. The invention is intended to make a contribution insofar that it would not be the measuring process that represents the rate-determining step; rather depending on the respective problem—optimum adaptation of the analysis to the test principle should be possible. If this does not succeed, rapid kinetics possibly cannot be treated in a multiplex format, or the reactions themselves will result in unspecific products, even if the reaction is more or less interrupted for measurement by a thermocyclic control.

The invention meets these requirements by providing a method on the basis of a new assay principle, a measuring system and a test assessment adjusted to the kit and measuring system.

According to the invention, said object is accomplished by providing a method for the multiplex analysis of a plurality of analytes.

According to the invention, such a method comprises the following steps:
a) use of a support which has at least two different microparticle populations immobilized thereon, said different microparticle populations differing in their fluorescence coding and at least two of the differently fluorescence-encoded microparticle populations essentially including microparticles occupied by a particular, specific acceptor molecule population, said acceptor molecule populations of said at least two differently fluorescence-encoded microparticle populations being different from each other;
b) measuring the fluorescence of the support from step a) with an optical resolution 1 prior to contacting the support with the sample to be analyzed,
   said resolution 1
      permitting differentiation of microparticle singlets, doublets, triplets, multiplets and monolayers, and
      allowing determination of the localized position of individual immobilized microparticles of the respective at least two different microparticle populations on the support, taking into account the different fluorescence coding of the at least two different microparticle populations;
c) contacting the support from step a) with the sample to be analyzed, the interaction of the respective analyte with the analyte-specific acceptor molecule on the corresponding immobilized microparticle causing a change in fluorescence (step c) may optionally be performed prior to step b));
d) performing at least one additional measurement of the fluorescence of the support during or after contacting in accordance with step c) with a resolution 2;
e) assigning the fluorescence values measured with resolution 2 to the individual microparticle singlets, doublets, triplets, multiplets and monolayers locally identified on the support in accordance with step b) and assigned to a particular acceptor molecule population;
f) determining the change in fluorescence for each locally identified microparticle singlet, doublet, triplet, multiplet and monolayer on the support by contacting in accordance with step c).

The method is based on the fact that microparticle populations can rapidly be made distinguishable in that these microparticles emit a population-specific fluorescence pattern upon excitation with suitable light. Using fluorescence-optical measurement, each microparticle can rapidly and unambiguously be determined in its localized position and arrangement on a suitable surface and assigned to a particular microparticle population. The method according to the invention uses as many distinguishable microparticle populations as analytes and controls must be detected in the sample to be analyzed. Each distinguishable microparticle population becomes occupied with molecules of a particular, specific acceptor molecule population, and each specific acceptor molecule population undergoes specific interaction with an analyte to be determined or is used as a control. At this point, each distinguishable fluorescence-encoded microparticle population is occupied with precisely one specific acceptor molecule population. The different microparticle populations, each one occupied with acceptor molecules of a particular specificity, are subsequently immobilized on a suitable support, which immobilization does not require a specific spatial arrangement of the different microparticle populations on the support. Following immobilization, a first measurement of the fluorescence of the loaded support is performed. This measurement is used as reference for future measurements in the method and, by virtue of the unambiguous fluorescence coding of the individual microparticle populations, subsequently allows precise spatial determination of the position of each single immobilized microparticle, its arrangement, e.g. as microparticle singlets, doublets, triplets, multiplets or as monolayers, and precise assignment of the respective particle to a particular microparticle population occupied with a specific acceptor molecule population. Thereafter, the support can be contacted with the sample to be analyzed. The specific interaction of the analyte with its acceptor molecule on the surface of the respective microparticle or with a particular ligand for this acceptor molecule results in a change in fluorescence. This change in fluorescence is measured in a single measurement or in a series of consecutive measurements. Using a positionally precise comparison of the reference measurement and following measurement(s) of sample, the change in fluorescence can be determined for each single microparticle and unequivocally assigned to a particular microparticle population.

For example, this method can be used to determine the concentration or affinity of analytes in a sample or establish reaction kinetics.

The individual steps and components of the method according to the invention will be described in more detail below.

### Multiplex Analysis

The preferred implemented multiplex level of analyses using the method according to the invention amounts to 2-1000 different analytes and controls per sample, in which event a plurality of measuring parameters such as different epitopes or nucleotide sequence sections of an analyte can be detected simultaneously. The preferred multiplex level amounts to 2-100 different analytes and controls per sample and more preferably 2-10 analytes and controls per sample.

### Analytes

Analytes can be any conceivable organic and inorganic molecules of a sample, which can be detected via their specific interaction with the acceptor molecules on the surface of the immobilized microparticles and the change in fluorescence caused in this way.

Preferred analytes are peptides, proteins, enzymes, lectins, antibodies, antigens, aptamers, polysaccharides, lipids or nucleic acids. In the event of nucleic acids, natural and synthetic DNA or RNA molecules are preferred, and nucleic acids formed in each cycle of amplification reactions such as PCR are particularly preferred. However, it is also possible to use non-natural nucleic acid analogs or amino acid analogs as well as molecules containing the same.

For example, the analytes to be analyzed can either be labeled themselves and directly cause a change in fluorescence via their label and their specific interaction with the acceptor molecule, or labeled or unlabeled analytes cause an indirect change in fluorescence in that the analytes, e.g. via their specific interaction with acceptor molecules or via specific interaction with a competing ligand for the specific acceptor molecule, influence or prevent binding of a correspondingly labeled ligand.

### Mix & Measure Test Format

Implementation of the test is facilitated in that the method according to the invention proceeds in the form of a mix & measure procedure. That is, all reactants are placed in a reaction vessel and, apart from sealing the reaction vessel, which may be necessary, measurement and evaluation, no further working steps are required.

### Supports

The reactions may proceed on planar supports or in wells of such supports and can be detected thereon. The supports can be in the form of slides, blisters, microtest plates, capillaries or tubes. Sealing of the wells can be effected using caps, sealed films, or by placing a layer on top. In a particularly preferred fashion, NucleoLink plates (Nunc) for nucleic acid amplification are used, which have a surface coating for covalent immobilization of biomolecules, a sufficiently planar and transparent vessel bottom and are thermally stable. Surface coatings can also be used to reduce non-specific binding of test molecules or detection reagents. Surface micropatterning which does not impair optical focusing is of course also possible.

The solid support can be in any form and can be made of various materials comprising in particular various metals, glass and plastic materials. Preferred solid supports are nylon membranes, epoxy glass and borofluorate glass. The advantage of using glass and plastic materials can be seen in the transparency of the materials, allowing the production of slide or microplate type supports for parallel high throughput of samples and cost reduction resulting therefrom. The microarrays can be in the form of a slide or microplate (also referred to as microtiter plate). A microplate is a dished container having a plurality of wells (at least two). Microplate-based microarrays involve a microplate with a plurality of wells having microarray biochips placed in the bottoms thereof. One example of a microplate is the well-known 96-well ELISA microtiter plate.

Furthermore, solid supports based on self-assembling layer systems are likewise suitable for implementing the present invention. Application may proceed using automatic methods.

The supports can be made of polycarbonate, polystyrene (other plastic materials), glass, metal, ceramics.

### Fluorescence-Encoded Microparticles

The availability of reactive or non-reactive additives of liquid plastic materials, e.g. thermoplastic materials, elastomers, thermoset materials, can be controlled efficiently by encasing or embedding in straight-chain or network-forming polymers. Polymer-based microcomposites of this type are known in the form of microcapsules with a core-shell structure or in the form of microscale matrix particles with a largely homogeneous distribution of the components over the particle cross-section (Ch. A. Finch, R. Bodmeier: “Microencapsulation” in Ullman's Encyclopedia of Industrial Chemistry, 6th Ed. 2001 Electronic Release). The core of microcapsules can be present in solid, liquid or gaseous form (hollow beads). In the event of matrix particles, systems with homogeneous phase and heterogeneous phase are known. According to the invention, all microparticles permitting immobilization are possible. The particles are preferably constituted of a polymer material. Preferred are the following polymer materials, including but not limited to polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, poly(vinyl acetate), poly(vinyl chloride), polyvinylpyridine, poly(vinylbenzyl chloride), polyvinyltoluene, poly(vinylidene chloride), polydivinylbenzene, poly(methyl methacrylate), polylactide, polyglycolide, poly(lactideco-glycolide), polyanhydride, polyorthoester, polysulfone or combinations thereof. Other polymer materials such as carbohydrates, e.g. carboxymethylcellulose, hydroxyethylcellulose, agar, gel, a proteinaceous polymer, polypeptides, eukaryotic and prokaryotic cells, lipids, metal, resins, latex, rubber, silicone, e.g. polydimethyldiphenylsiloxane, glass, melamine, ceramics, charcoal, kaolinite, bentonite and the like can be used in the same manner. Also, a magnet or a magnetically responsive metal oxide selected from the group of superparamagnetic, paramagnetic or ferromagnetic metal oxides can be incorporated in these polymers. The particles may have additional functional groups on the surface, such as carboxylates, esters, alcohols, carbamides, aldehydes, amines, sulfur oxides, mercapto groups, nitrogen oxides or halides, which can facilitate binding of analytic reactants and/or particle-particle binding.

Methods of producing microparticles by means of reactive and non-reactive particle formation processes have been described many times. In the event of reactive particle formation, the formation of wall or matrix is effected in parallel with a polymerization, polycondensation or polyaddition process. In the event of non-reactive methods, film-forming polymers are used directly and caused to undergo thermodynamic phase separation and particle formation. Methods of encapsulating solid or liquid core materials utilize e.g. melamine-formaldehyde resins. In particular, melamine-formaldehyde resins can be used for many purposes and without problems and can be applied from an aqueous phase for particle formation. The microcapsule size can be adjusted depending on the reaction conditions, e.g. addition of emulsifier or method of dispersing, and is between 0.5 and 30 μm for the use according to the invention. Another option is hydrothermal crosslinking of hydroxymethylmelamine.

Fluorescence-encoded microparticles are well-known to those skilled in the art and have been described e.g. in DE 699 07 630 T2 and DE 10054382.0. The dyes, if more than one dye is used to stain more than one population of particles, are selected in such a way that that they have essentially different emission spectrums, preferably have emission maximums separated by more than 10 nm, more preferably emission maximums separated by more than 25 nm and even more preferably by more than 50 nm. The dyes can be selected to have emission bands matching the commercially available filters, or for the detection of multiple fluorophores with different excitation and emission bands. Insert list of classes of fluorescent dyes here.

Fluorescent dyes used to label the microparticles are all substances capable of emitting detectable luminescence signals. However, it is also possible to use dyes which emit X-radiation or exhibit phosphorescence. Fluorescent dyes in the meaning of the invention are all those gaseous, liquid or solid inorganic and/or organic compounds that are characterized in that, after excitation, they re-emit the absorbed energy in the form of radiation of equal, longer or shorter wavelength. That is, inorganic or organic pigments or “quantum dots” capable of providing luminescence can also be used as fluorescent dyes in the meaning of the invention. However, it can also be envisaged that the microparticles are such in nature that they exhibit autofluorescence or have both autofluorescence and a non-autofluorescent label. For example, autofluorescence of the microparticles can be generated by including the mineral fluorite in the microparticles. For example, dansyl chloride, fluorescein isothiocyanate, 7-chloro-4nitrobenzoxadiazole, pyrenebutyrylacetic anhydride, N-iodoacetyl-N′-(5-sulfo-1naphthyl)ethylenediamine, 1-anilinonaphthalene-8-sulfonate, 2toluidinonaphthalene-6-sulfonate, 7-(p-methoxybenzylamino)-4-nitrobenz-2-oxa-1,3-diazole, formycin, 2-aminopurineribonucleoside, ethenoadenosine, benzoadenosine, α- and β-parinaric acid and/or Δ9,11,13,15-octadecatetraenic acid, cadmium selenite crystals of a single or different sizes etc. can be used as non-autofluorescent dyes. Likewise, e.g. transition metal complexes containing the following substances can be used as fluorescent dyes: ruthenium(II), rhenium(I) or osmium and iridium as central atom and diimine ligands; phosphorescent porphyrins with platinum, palladium, lutetium or tin as central atom; phosphorescent complexes of rare earths such as europium, dysprosium or terbium; phosphorescent crystals such as ruby, Cr—YAG, alexandrite or phosphorescent mixed oxides such as magnesium fluorogermanate or cadmium selenite crystals, fluorescein, aminofluorescein, aminomethylcoumarin, rhodamine, rhodamine 6G, rhodamine B, tetramethylrhodamine, ethidium bromide and/or acridine orange. For example, the following combinations of substances can be used as fluorescent dyes:
ruthenium(II)-tris(4,7-diphenyl-1,10-phenanthroline)/HPTS
ruthenium(II)-tris(4,7-diphenyl-1,10-phenanthroline)/fluorescein
ruthenium(II)-tris(4,7-diphenyl-1,10-phenanthroline)/rhodamine B
ruthenium(II)-tris(4,7-diphenyl-1,10-phenanthroline)/rhodamine B octadecyl ester
ruthenium(II)-tris(4,7-diphenyl-1,10-phenanthroline)/hexadecylacridine orange
europium(III)-tris-theonyl-trifluoromethylacetonate/hydroxymethylcoumarin
platinum(II)-tetraphenylporphyrine/rhodamine B octadecyl ester
platinum(II)-tetraphenylporphyrine/rhodamine B
platinum(II)-tetraphenylporphyrine/naphthofluorescein
platinum(II)-tetraphenylporphyrine/sulforhodamine 101
platinum(II)-octaethylporphyrine/eosin
platinum(II)-octaethylporphyrine/thionine
platinum(II)-octaethylketoporphyrine/Nile blue
Cr(III)-YAG/Nile blue and
Cr(III)-YAG/naphthofluorescein.
aminocoumarin/aminofluorescein
aminocoumarin/rhodamine 6G
aminocoumarin/tetramethylrhodamine
aminocoumarin/acridine orange
aminofluorescein/rhodamine 6G
aminocoumarin/Nile blue
aminofluorescein/tetramethylrhodamine
aminofluorescein/ethidium bromide and
DAPI/rhodamine.

However, it is also possible to use combinations of 3 dyes for encoding the particles, e.g.:

aminocoumarin, rhodamine, Nile blue, or
aminocoumarin, fluorescein, rhodamine. Other combinations are also possible.

What might be referred to as surface fluorescence is either the fluorescence of the outermost layer of the microparticles or, if the acceptor molecules on the particle surface are fluorescence-labeled themselves, the fluorescence of said acceptor molecules.

What is referred to as ligand fluorescence is the fluorescence which either originates from an analyte labeled with a corresponding dye or, if a correspondingly labeled, competing ligand is employed, the fluorescence of said labeled ligand, or when a fluorescent detection molecule binds to the ligand that is bound to the acceptor molecules.

In a preferred variant one or more dyes from the class of coumarins, rhodamines or Nile blue derivatives are incorporated by polymerization in the particles for particle coding, and a fluorescein derivative is used as surface fluorescence or to label ligands.

If coumarin, fluorescein and rhodamine are used for coding, Cy5 and fluorescent dyes of higher emission wavelength can be used for surface fluorescence or ligand fluorescence. However, it is also possible to implement the surface fluorescence or ligand fluorescence via a FRET pair such as fluorescein and rhodamine. In this event, coumarin and cyano dyes etc. are available for coding. The options of coding increase enormously in that the same dye can be employed at different concentrations in a plurality of layers of the microparticles. This results in a wide variety of possible ratios being formed for each layer and between different layers, which can be utilized for decoding of the particle populations.

Another possible way of creating structure within microparticles is layered polymeric incorporation of additional fluorescent or fluorescence-encoded microparticles. In this way it is possible to create patterns in the microparticles in a specific manner, e.g. coarse or fine granulation or number of granules per layer, which can be utilized for coding a greater number of microparticle populations.

According to the invention, microparticles of melamine, silica, polysulfone and polyether, polymethacrylate are preferably used, with polymethacrylate being particularly preferred. As a rule, they have a diameter of from 0.5 to 60 μm. The preferred particle size is 5 to 15 μm. The shape of the particles can be spherical, elliptical, cylindrical, irregular or cuboid.

The microparticles preferably used are remarkable in that they are constituted of preferably 2 layers and a core which are different in fluorescence-optical terms, at least the outer layer being resistant to temperature and consisting of e.g. melamine. Preferably, the central and inner layers are likewise constituted of a temperature-resistant polymer, in case the tests being used should proceed at elevated temperatures. The particles may include magnetic particles in one or more layers, by means of which reliable immobilization of the particles during measurement is ensured.

The different layers may include one or more fluorescent dyes used for coding, as well as fluorescent dyes coupled to the particle surface either directly or mediated through biomolecules. Fluorescent dyes bound to the surface via biomolecules may therefore assume the function of coding as well as the function of indicating binding or reaction kinetics.

As an alternative to fluorescence-encoded particles it is also possible to use cells in the form of suspensions, monolayers or tissue sections, in which event different cell populations can be distinguished via their fine structure or optionally via their fluorescence coding.

The polymers on the particle surface can be in unmodified form or in modified form. Possible functionalizations are carboxy, sulfohydryl, epoxy, amino, hydroxy, sulfonic acid, pegylyl, acrylic, phosphate groups.

### Microparticle Immobilization

It is preferred to use a device including fluorescence-encoded microparticles as points of measurement, which microparticles bear acceptor molecules and are immobilized by gravity, magnetic or mechanical force, via chemical or physical binding to the vessel bottom either permanently or during the time interval of the measuring process. The microparticles can be coupled to the support either directly or mediated through linker molecules. The immobilized acceptor molecules, preferably representing biomolecules, likewise act as linker molecules. The immobilized particles of the different particle populations undergo random distribution over the intended area of the support and can be in the form of singlets, doublets, triplets, multiplets, complex arrangements and even monolayers. The most important function of particle immobilization is the advantage that decoding of the particle fluorescences must be performed only once and merely the surfaces or the ligand fluorescence must be inspected thereafter to record further points of measurement. If the particles are not permanently immobilized, the particle fluorescences must also be collected during recording further points of measurement.

The particles need not be firmly immobilized on a solid surface but rather must be held stably in a focal plane, which can also be ensured by means of a density gradient. Thus, when using particles of different densities, additional coding of particle populations can be achieved. For example, different densities can be obtained by using different polymers in the preparation of particles and particle layers. The structure of the gradient is ensured e.g. by simultaneous pipetting of sample and density gradient, where the dilution of the sample can be constant or may vary across the gradient. When varying the sample dilution it is possible e.g. to perform a titration of the analyte in a reaction batch. The density gradient can be in the form of a homogeneous gradient or a step gradient. The particles can undergo sedimentation to the desired phase boundary through gravity. In the event of a charge gradient, preferably adjusted using ampholytes, the particles can also migrate to their isoelectric point in accordance with their charge and through the action of an applied field. By combining homogeneous charge and density gradients, it is possible to create bead multilayers in the reaction vessel. Advantageously, a planar focal plane is achieved by virtue of the phase boundaries between different densities despite the fact that the bottoms of the reaction environment are non-planar—e.g. the bottoms of microtest plates are never ideally planar. This saves efforts in focusing and thus time. It is also possible to enclose the particles with the reaction solution between an apolar liquid of lower density and an apolar liquid of higher density to thereby confine the reaction volume. It is also possible in this way to minimize the background fluorescence. Background fluorescence can also be minimized by adding the reaction solution with e.g. a quencher dye or light-absorbing nanoparticles which do not impair the reaction but increase the contrast and thus the sensitivity. In a preferred fashion such substances are added to increase the contrast, which substances correspond to the light of the excitation wavelength and/or emission wavelength or the ligand fluorescence.

### Acceptor Molecules

Substances specifically binding the analyte from a sample or reference sample with high sensitivity and specificity or competing with the analyte for binding of an analog are used as acceptor molecules which are preferably biomolecules. The acceptor molecule and the analog or ligand may bear a fluorescent label and/or a quencher. The immobilized biomolecules can be peptides or proteins and, preferred herein, antibodies, antigens, enzymes, lectins, aptamers. However, they can also be polysaccharides, lipids or nucleic acids. In the event of nucleic acids, synthetic DNA or RNA oligonucleotides are mainly used. However, it is also possible to use non-natural nucleic acid analogs.

Coupling of the acceptor molecules to the microparticles is covalent and/or noncovalent.

### Change in Fluorescence

The reactions necessary for detection are intermolecular interactions proceeding at the boundary surface between the solid phase of the particle surface and the liquid phase of the reaction solution. Depending on the test principle, this results in a change of the surface or ligand fluorescence, which becomes apparent via an increase or decrease of the surface or ligand fluorescence intensity, polarization, lifetime on the particle surface and is recorded over time using at least two points of measurement. A reduction of the fluorescence intensity can occur in such a way that, as described above, fluorescence-labeled biomolecules immobilized on the particle surface are degraded during reaction.

Alternatively, an increase of the fluorescence signal may occur when a quencher is displaced from its spatial proximity to the quenched fluorophore as a result of the reaction, so that the fluorophore becomes fluorescent as is the case with e.g. molecular beacons, scorpions, amplifluor primers. Quenching of the fluorophore can also be terminated by degradation of an immobilized TaqMan probe during the reaction, so that the fluorophore after degradation of the probe remains on the particle surface and fluoresces. Also, FRET pairs may form as a result of spatially close binding of fluorescent dye molecule-labeled probes to the analyte molecules, which in turn have been immobilized on the surface of the particles during the reaction, e.g. by means of PCR. It is also conceivable that the fluorescence signals of ligand fluorescence on the particle surface is increased by binding of fluorescence-labeled detection molecules such as anti-phosphate group-specific antibodies. As a result of binding to peptides phosphorylated and immobilized on the particle surface during the reaction, fluorescent groups are measurably increased in their concentration without requiring quenching and FRET systems.

The change in fluorescence to be measured can preferably be:
a decrease of the surface fluorescence, e.g. as a result of degradation;
an increase of the surface fluorescence, e.g. by displacement of a competing quencher-labeled ligand from the acceptor molecule, which is labeled with a suitable dye, by one or more analytes;
a decrease of the ligand fluorescence, e.g. as a result of specific binding of an analyte labeled with a suitable dye to a quencher-labeled acceptor on the particle surface;
an increase of the ligand fluorescence, e.g. as a result of specific binding of an analyte labeled with a suitable dye to a corresponding acceptor molecule on the microparticle surface;
a change of the overall fluorescence, e.g. via combined action of a FRET pair of acceptor molecule and analyte.

Examples of reactions are PCR, solid-phase PCR, isothermal nucleic acid amplifications such as strand displacement amplification or ligase chain reaction or other enzyme reactions, binding of antibodies to antigens.

### Measuring System

Devices for the inventive implementation and measurement of changes in fluorescence on bead arrays of a variety of test formats which can be controlled fully automatically by means of a computer comprise:
a fully automatic control by means of a computer;
a thermocycler for rapid temperature control of samples with a heating/cooling rate of 3-20° C. per second, which has a reaction section with a plurality of temperature-controllable receiving means for supports in accordance with claim 1, step a);
a positioning means for the thermocycler and the reaction environment, which can be controlled via thermocycles;
an illumination means associated with the reaction section, by means of which excitation light of varying, defined wavelengths can be radiated;
an optical means preferably suitable for everse and inverse fluorescence detection using appropriate optical filters; one or more detector means (e.g. CCD, CMOS) generating images depending on a measured fluorescence intensity; and
an evaluation unit which generates measurement values from the images.

When using microparticles, the devices may comprise additional immobilization means for e.g. magnetic or paramagnetic particles.

By using optically transparent vessel bottoms and/or vessel caps with low autofluorescence, which permit fluorescence-optical evaluation during the thermocyclic or isothermal implementation of the detection reaction, measurements with incident light and transmitted-light fluorescence are possible. In the event of incident light fluorescence, commercially available microtiter plates such as NucleoLink or NucleoSorb can be used. The commercially available reaction vessels used can be coated to reduce the autofluorescence.

When using non-permanently immobilized microparticles, all particle fluorescences must be measured when collecting additional points of measurement so that great demands on the speed of the hardware are to be made, e.g. use of preferably low resolution of the lenses at high speed, use of multiband filters to avoid filter changing, in combination with multicolor LED illumination, use of an electron-amplifying camera, parallelization of the detector units. Detector units can preferably be arranged in lines of 8, in the sense of a parallelization, or focused on a camera through glass fiber or liquid conductors. Also, simultaneous measurement of a number of wells is possible by means of wide-angle lenses. The time for measuring can also be reduced by pixel binning because the sensitivity increases, so that exposure is shorter and the amount of data is reduced.

### Collecting Points of Measurement

As envisaged in the invention, a first point of measurement is recorded prior to or at the beginning of the reaction, and another point of measurement is recorded during the further course or at least after terminating the reaction so as to allow an estimation concerning the extent of reaction for an individual analyte or the concentration of analyte in the sample.

Recording the first point of measurement is necessary to identify all microparticles possible for evaluation, which is done by acquiring the coding fluorescences, so as to allow assignment of all particles to a particular particle population. Further, the x/y position of a required number of identified particles per population necessary for evaluation is stored to allow retrieval of the sites of measurement for recording the surface or ligand fluorescence when acquiring additional points of measurement. Also, the surface or ligand fluorescence is recorded as a reference for the surface or ligand signal recorded in the following points of measurement and optionally used for decoding of the particle population. The fluorescences are recorded using an optical resolution 1 which usually is higher than resolution 2 to reliably record the evaluable units, preferably single immobilized particles, but also doublets, triplets or monolayers of particles, if necessary. By acquiring images in a number of Z planes and subsequently calculating an overall definition image (“extended depth of field” algorithms), doublets and higher agglomerates can be used to reliably determine the single-bead boundary, thereby achieving singularization.

Alternatively, agglomerates that have been recognized are divided into separate groups according to their area and morphological properties. For each group a special algorithm variant is used to calculate the fluorescence. The beads can be digitally singularized by determining the bead contact points.

Alternatively, ring structures in an image that are overlaid by other beads can be supplemented by bead extrapolation using a priori knowledge (a bead is e.g. a sphere). In this way it is possible to evaluate multilayer arrangements of beads (one algorithm from the literature is the Hough-Circle transformation). It is always the visible area of a bead that is assessed.

Alternatively, single beads can be extracted from the image data of bead agglomerates by digital quenching of single bead populations. These alternatives can be employed separately or in combination so that more evaluable particles per recorded image are available, which in turn saves time for analysis.

Collecting all further points of measurement is performed using resolution 2 and is preferably confined to recording the surface or ligand fluorescence. In another embodiment it is possible to acquire particle fluorescences as well in order to document precise retrieval of particles for at least a few measuring points. If a single fluorescence of the particle fluorescence radiates slightly but reliably detectably into the emission channel of the surface fluorescence, the sensitivity will be reduced, but the particle can be retrieved. It is also possible to use two surface fluorescences, one of them not being subject to a change in fluorescence during the reaction, so that particle shift due to e.g. thermal effects can be ruled out. The resolutions used must be optimally adjusted to the particle density and the number of pixels per particle (1-1000 pixels) so as to ensure the reliability in identifying and retrieving the particles which is required in each use.

Recording said further points of measurement can be effected prior to, during and after the reaction at regular time intervals, or after a particular temperature or a particular section of a thermocycle has been reached as a result of temperature control. In a preferred fashion the measurements are performed during an isothermal part of the reaction so as to minimize influences as a result of temperature fluctuations, such as expansion of material and increased convection in the reaction mixture.

In addition to recording the surface or ligand fluorescence, it may be advantageous to record a particle fluorescence from time to time, because decreasing surface fluorescence involves a risk in that a particle may not be retrieved properly. To minimize bleaching, preferably when using LED and especially when recording further points of measurement, the illumination intensity can be adjusted to the surface fluorescence. The varied illumination intensity should be referenced, if necessary (reduction of bleaching by limiting the illumination to the imaging period). Furthermore, reducing the lens magnification allows imaging of a larger measuring area, so that a smaller number of images per well are required. Avoiding secondary focusing and secondary exposure by means of algorithms such as extended depth of field (increasing the depth of field by adding up a plurality of images in Z) and “high dynamic range” (increasing the dynamics of exposure by adding up a very bright and a very dark image) likewise reduces the measuring time.

1-1000 particles of a population are acquired per measuring point, preferably 1-100 particles, and in a particularly preferred embodiment 1-10 particles are acquired.

### Analysis

### Assignment of Measuring Points to Particles and Particle Populations

For decoding of the particle populations it is preferred to use the fluorescences of dyes incorporated by polymerization in all particles or of dyes incorporated by polymerization in different layers. These different fluorescence intensities are used to calculate ratios which permit unambiguous characterization of each particle population

Thus, the fluorescence can be determined in the form of a determination of the fluorescence intensity of a particular emission wavelength, recognizable as an annular fluorescence in the particle periphery, prior to beginning a nuclease reaction for the determination of the particle population, but also for determining the maximum signal of the surface fluorescence of the respective particle prior to the reaction. This maximum signal declines during the course of the reaction because the biomolecules undergo enzymatic degradation and, as a consequence, the dye is released from the particle surface. Indeed, the dye is no longer available for decoding of the particle populations, but decoding is no longer required because the particle has been immobilized. To control proper particle retrieval without decoding, the fluorescences of the dyes incorporated in the particles by polymerization are sufficient in this case. Coding fluorescences, surface and/or ligand fluorescences can also be used to reference particles of different sizes with respect to detected fluorescence intensities.

### Calculation of Kinetics

The analysis of the change in fluorescence as a result of reaction from measuring point to measuring point furnishes reaction kinetics from which concentrations of the analytes or binding constants can be calculated. If assessment is related to a reaction cycle, as in a polymerase chain reaction, the indication of the breakthrough cycle where the signal or change of the signal breaks through the background is decisive as a measure of the amount of reaction product formed. In this way a much more accurate quantification of PCR products in a sample is possible as compared to determining the fluorescence intensity.

Referencing all further measuring points to measuring point 1 or one of the following points of measurement prior to or at the beginning of the reaction substantially reduces measuring errors because influences such as varying illumination or particle size can be minimized through the option of referencing to a particle population as a positive control or to a reference sample.

The method also envisages the necessity of systematically acquiring the influence of disturbance variables so as to be able to take account thereof when computing the measurement values. Such disturbance variables can the bleaching/fading of a number of fluorescent dyes as well as influences of temperature fluctuations on measurement or unspecific background fluorescence.

### Kit

The present invention also relates to a kit for use in a method according to the invention, said kit including preformulated reagents comprising at least one microparticle population coated with acceptor molecules.

Using the system according to the invention it is possible to provide a prefabricated real-time PCR kit which includes all required ingredients so that separate pipetting of buffers, nucleotide mix, reporter molecules, polymerases, reverse transcriptases and primers and optionally anti-bleaching agents is not necessary. For quantification, this (traditional) method of real-time PCR utilizes melting point determination of amplified DNA and analysis of the threshold value of the fluorescence intensity (CT).

The method according to the invention can be used in:
qualitative and quantitative nucleic acid multiplex amplification,
nucleic acid sequence screening,
immunodetection to determine the concentration of antigens and antibodies or corresponding binding constants and determine enzyme activities as well as intermolecular interactions between proteins, proteins and low-molecular weight substances, proteins and nucleic acids, carbohydrates, lipids.

### Advantages:

Referencing solely to measuring point 1 and optionally to a comparative sample. In this way the measurement is made more precise and it is possible to use or measure less particles per population.
Localization and decoding are necessary only once, thereby saving time and achieving a reduction of data.
Lower resolution 2 contributes to the ability to record a large area so that more particles per recording operation are detected, which is time-saving.
A mix & measure assay is easy to handle so that the number of possible errors is low. In addition, it is possible to acquire reaction kinetics, thereby extending the quality of analysis.
Reducing the number of particles per population required for evaluation not only allows saving material and time, but also measurement of more populations per unit area, thereby increasing the possible multiplex level.
By using 2 different resolutions it is possible by means of a higher resolution to identify and localize all evaluable particles prior to beginning the reaction, which is time-consuming. Resolution 2 can be utilized to acquire the surface fluorescence or ligand fluorescence of other points of measurement because only those points of measurement have already been acquired with resolution 1 that are reliably retrieved with resolution 2 when acquiring later points of measurement. The lower resolution 2 allows to acquire more particles in a larger measuring area on the support, thereby substantially speeding up the measuring process.
Filters need not be changed when recording additional points of measurement for a single fluorescence emission of the surface or ligand fluorescence, which saves time.
Dimensioning of the detection of these various parameters with respect to time and space makes the test immensely robust compared to conventional test systems.

Without intending to be limiting, the invention will be explained in more detail below with reference to the drawings and examples.

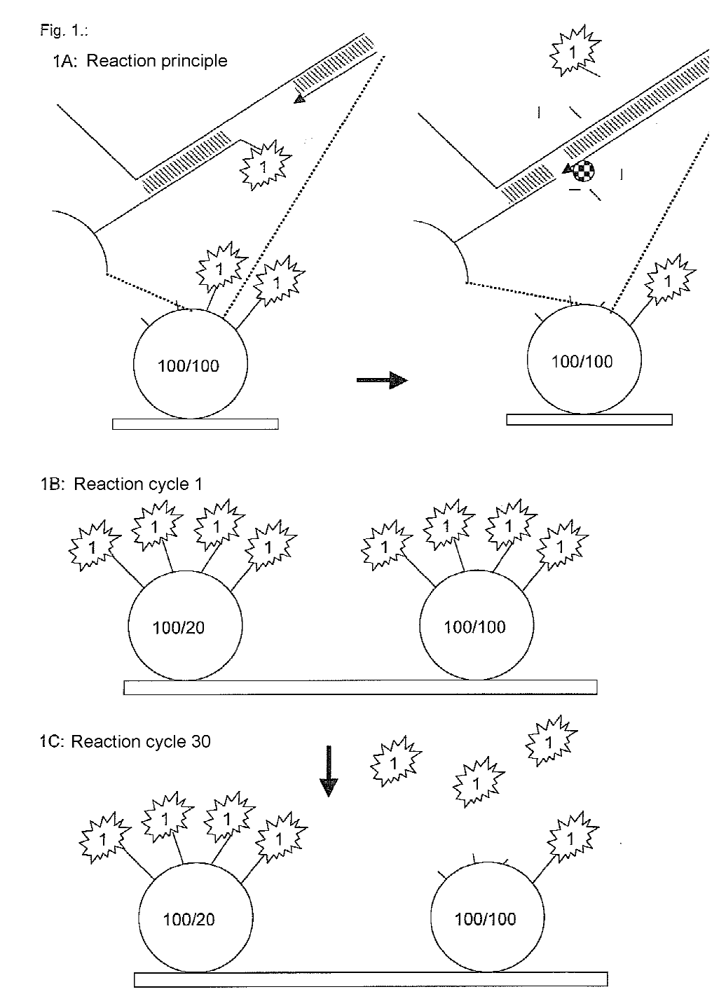

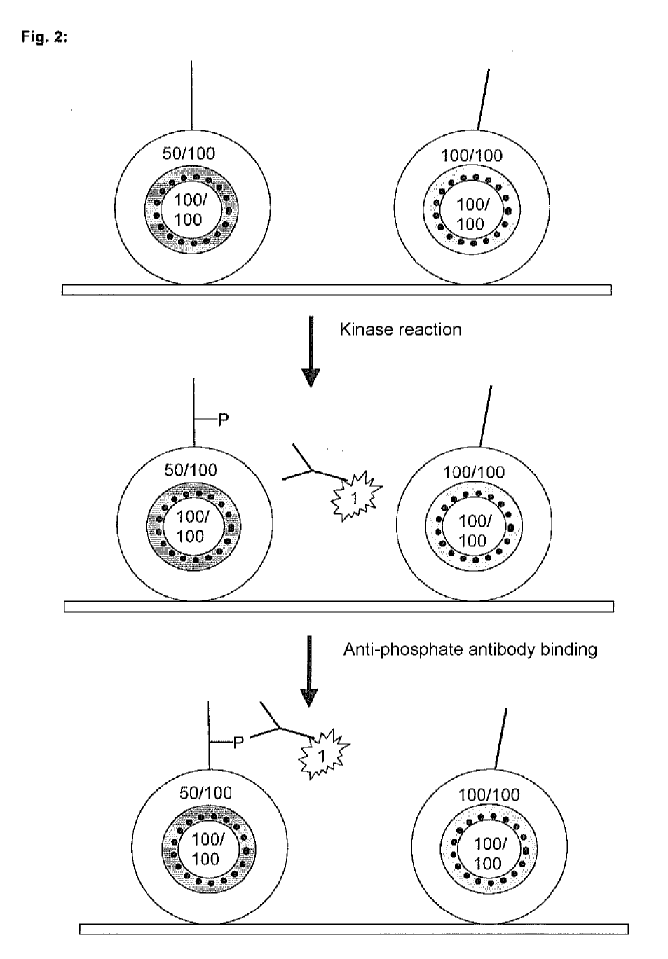

### FIG. 1

### Schematic Representation of the Detection of a Plurality of Target Sequences Using a Combination of Real-Time PCR and Microparticle Array

To detect amplificate having different target sequences, a multiplex PCR is performed in the wells of a NucleoLink plate, combined with simultaneous detection of the amplificates on a bead array. The thermally stable microparticles of the bead array, encoded with 2 fluorescent dyes (quantity ratios 100/20 and 100/100), are immobilized at the bottom of the NucleoLink plate and bear one amplificate sequence-specific capture probe per microparticle population, which has been labeled with a surface fluorescence. During the PCR the amplificate molecules hybridize to the capture probes which, when using a DNA polymerase with 5′-3′ exonculease activity, are degraded complementarily to the hybridized amplificate strand during the next primer extension (FIG. 1A). During this process the surface fluorescence is released into the reaction medium, resulting in a reduction of the surface fluorescence on the particle surface during the course of several reaction cycles (FIGS. 1B, 1C). Capture probes for which no complementary target sequence has been amplified will not be degraded so that the surface fluorescence is retained, taking account of possible bleaching effects. An amplified sample is positive for a target sequence if the corresponding particle population has lost more surface fluorescence during the reaction than an internal or external standard sample. In particular, the use of internal standards has proven to be a very reliable procedure.

Instead of surface fluorescence it is possible to use reporter systems such as FRET probes which cause an increase in ligand fluorescence when the amplified product is bound to the capture probes.

For example, the invention is utilized in an array as follows:

particles of particle population 1 (left part of Figure):
the particle includes e.g. 100 parts of aminocoumarin and e.g. 20 parts of Nile blue (100/20).
Particles of particle population 2 (right part of Figure):
the particle includes e.g. 100 parts of aminocoumarin and e.g. 100 parts of Nile blue (100/100).
Dye 1 for the surface fluorescence is e.g. rhodamine.

After contacting the amplificate-containing sample with the immobilized microparticles or cyclic formation of the amplificate during amplification in the same reaction vessel, the particle fluorescences and, if used for coding, the surface fluorescence are recorded using microscopic lenses. To this end, the aminocoumarin mentioned as example is excited at a wavelength of 350 nm to detect the first coding fluorescence of the particles at 445 nm. Subsequently, e.g. rhodamine is excited at a wavelength of 530 nm and measured at 560 nm to determine the surface fluorescence. Using subsequent excitation at 680 nm and detection at 720 nm, the Nile blue mentioned as example is excited and detected to determine the second coding fluorescence of the particle.

For decoding of the particle codings, computing the fluorescence signal detected in a spatially resolved manner proceeds according to the following rules:
Determination of quotient 1 of aminocoumarin and Nile blue fluorescences
   In the presented example the following ratios are obtained:
   particle population 1: 5
   particle population 2: 1

The particle fluorescences are recorded especially on densely packed particles, e.g. in those cases where many parameters must be detected in the presence of high multiplex levels, preferably at resolution 1, using a lens with 20-times magnification. The rhodamine surface fluorescence is preferably recorded between the reaction cycles 20 and 35, in which event measurement need not be effected in each cycle so as to save time and avoid bleaching effects. The surface fluorescences are preferably recorded with resolution 2, using a lens with 4-times or 10-times magnification. Signals of unambiguously identifiable singlets are preferably used in assessment.

The amplificate fluorescence, e.g. the one corresponding to the emission of the rhodamine FRET signal, can be output as an intensity or referenced versus a parameter maintained constant, such as the particle overall diameter which can be acquired e.g. via the rhodamine signal. If the surface fluorescence of additional measuring points drops below the value of e.g. the triple standard deviation of the maximum signal minus bleaching effects, the sample is deemed to contain target sequence. More specifically, determination of the threshold cycle allows precise multiplex quantification of target sequences.

### FIG. 2

### Schematic Representation of Multiplex Detection to Characterize Kinase Activities

Multiplex detection to characterize kinase activities in a sample is based on a bead array wherein fluorescence-encoded microparticles bear phosphorylatable peptides that are contacted with samples containing a corresponding kinase. In the event of peptide phosphorylation, a fluorescence-labeled anti-phospho group antibody binds to the phosphate groups, thereby concentrating ligand fluorescence on the particle surface. The ligand signal generated on the particle surface in this way is monitored for a number of measuring points. The signal can also be generated via FRET between a fluorophore 1 on the peptide and a fluorophore 2 on the anti-phospho group antibody.

The microparticles used are remarkable in that they are preferably constituted of 2 layers and a core which differ in fluorescence-optical terms. The central layer optionally includes particles, such as magnetic particles, by means of which reliable immobilization of the particles during a measurement or patterning of the respective layer can be ensured.

For example, the invention is utilized in an array as follows:

Particles of particle population 1 (left part of Figure):
The core includes e.g. 100 parts of aminocoumarin and e.g. 100 parts of Nile blue (100/100). The central layer includes e.g. 100 parts of fluorescein and the outer layer includes e.g. 50 parts of aminocoumarin and e.g. 100 parts of Nile blue.
Particles of particle population 2 (right part of Figure):
The core includes e.g. 100 parts of aminocoumarin and e.g. 100 parts of Nile blue (100/100). The central layer includes e.g. 100 parts of fluorescein and the outer layer includes e.g. 100 parts of aminocoumarin and e.g. 100 parts of Nile blue.
Dye 1 on the anti-phospho group antibody is e.g. fluorescein or rhodamine.

The particles of peptide-coated microparticle populations and the reaction mixture consisting of buffer solutions and anti-phospho group antibodies are placed in the well of a microtest plate. All microparticles in the batch, unless they have not been previously stably immobilized on the bottom of the microtest plate, are sedimented or immobilized on the vessel bottom using a permanent or electric magnet.

Using fluorescence spectroscopy, all fluorescences of the microparticle populations are recorded in association with point of measurement 1, the micropopulations are identified, and the position of the particles in the reaction vessel is determined. To this end, the aminocoumarin mentioned as example is excited at a wavelength of 350 nm to detect the first coding fluorescence of the particles in the outer layer and inner layer. Subsequently, e.g. fluorescein is excited at a wavelength of 480 nm and measured at 520 nm to determine the second coding fluorescence of the central layer of the particles and the ligand fluorescence. Using subsequent excitation at 680 nm and detection at 720 nm, the Nile blue mentioned as example is excited and detected to determine the second coding fluorescence of the core or outer layer.

Computing the fluorescence signals detected in a spatially resolved manner proceeds according to the following rules:

### Core:

Determination of quotient 1 of aminocoumarin and Nile blue fluorescences
Determination of the core diameter

### Central Layer:

Determination of quotient 2 from the fluorescein fluorescence and aminocoumarin or Nile blue fluorescence. This is possible because the shells partially overlap as a result of the particle structure, although the middle shell has only fluorescein incorporated therein by polymerization.
Determination of the thickness of layer 2

### Outer Layer:

Determination of quotient 3 from the aminocoumarin and Nile blue fluorescences
Determination of the thickness of layer 3

According to these rules, the microparticles of the present example can be distinguished in that quotient 3 of the two particle populations differs measurably.

### Quotient 3

Particle population 1: 0.5
Particle population 2: 1

All the other quotients and all the other diameters and layer thicknesses of the particles do not differ from each other.

Point of measurement 1 is recorded with resolution 1 using a lens with 4-times magnification. The other measuring points used to determine the development of the ligand fluorescence are likewise recorded with resolution 1 using a lens with 4-times magnification. For proper retrieval of the particles, the relative spatial coordinates of the particles from measuring point 1 are used to allow compensation of inaccuracies when approaching the measurement areas several times. For this reason, it is particularly preferred in the assessment to include particles immobilized as singlets because identification, decoding and retrieval thereof is easier and more reliable. Previously quantified bleaching effects of the fluorescent dyes used must be corrected before deriving the ligand fluorescence values from the data. The increase as well as the rapidity of the increase in ligand fluorescence over different points of measurement are proportional to the kinase activity in the sample (see also Example 2).

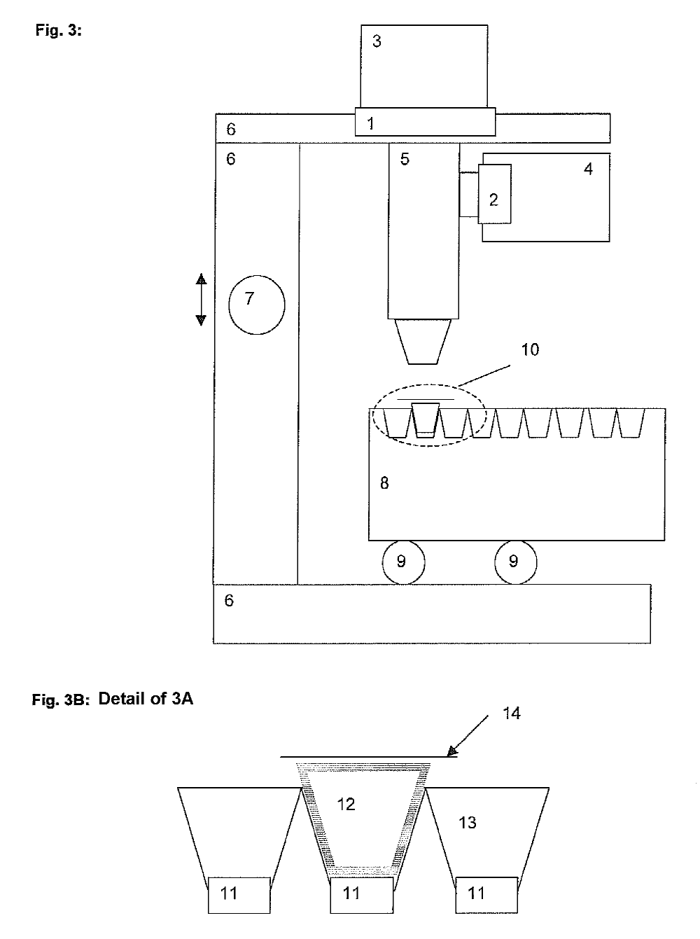

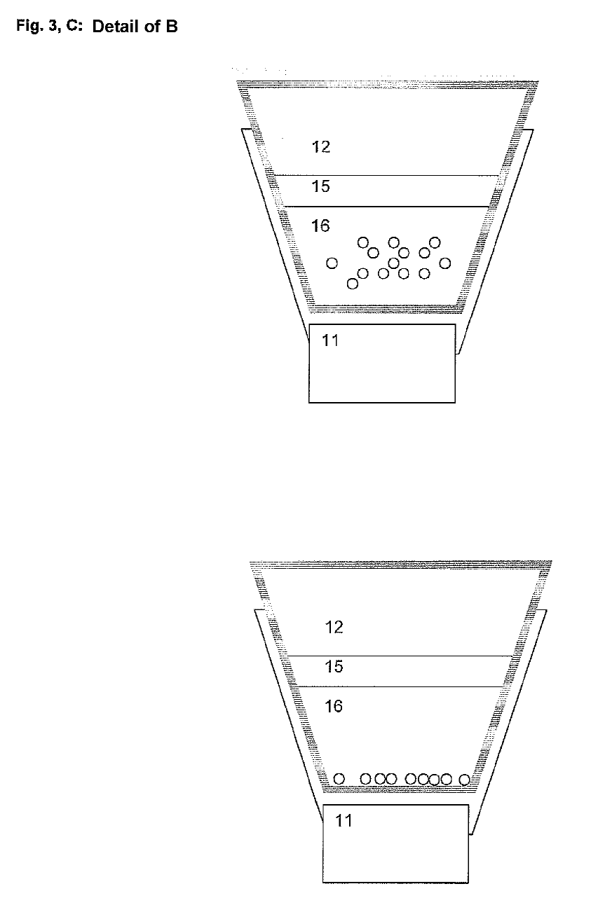

### FIG. 3

### Schematic Representation of an Apparatus System (A) and a Reaction Environment (B) for Combined Amplification of Target Sequences and Detection of Amplificates on a Microparticle Array

(A) With respect to its performance parameters, the fully automatically computer-controlled device represents a combination of an everse fluorescence microscope and a thermocycler.

A stand 6 with a motor z drive supports the optical components consisting at least of a long-distance optical system 5 for fluorescence detection, equipped with a 20× lens and elements for ray guiding and image generation. Excitation is effected by means of at least one xenon lamp or at least two different LEDs 3 or lasers 4, preferably in the spectral ranges of 350-400 nm, 450-500 nm and 550-600 nm. The concrete wavelengths of the excitation light depend on the fluorescent dyes used and are finely adjusted by means of suitable filters 2, if necessary. The light emitted by the fluorescent dyes is separated from the excitation light by means of suitable emission filters 1 and recorded with a sensitized camera 3. The stage has an x/y drive with an accuracy of <0.1 mm and accommodates the thermocycler with a sample holder (in this case for 96 reaction vessels with a capacity of 0.2 ml). A magnet 11 (permanent magnet or electric magnet) is optionally positioned beneath the reaction vessels with planar bottoms so that paramagnetic microparticles are properly immobilized during the measuring operation.

An automatic computer control embraces the PCR program and thus the temperature profile of the PCR, the filter changer, camera, image acquiring and image processing devices, the x/y/z drive, excitation light control, as well as the user interface, so that the output of amplificate fluorescences for the different particle populations can be ensured during the PCR.

As an alternative to the everse fluorescence microscope, the device may also be configured as an inverse fluorescence microscope.

(B) For example, NucleoSorb 8-packs 12 sealed with a cover glass are used as reaction vessels.

(C) The PCR master mix, together with sample DNA 16, is pipetted into the reaction vessel and overlaid with oil. Thereafter, the reaction vessel is optionally sealed with a cover glass and the PCR/measuring operation is started. As the PCR cycles proceed, the fluorescences are measured preferably during the phase of annealing (attachment of primer) under isothermal conditions. Alternatively, a melting curve is recorded at the end of the PCR. During the PCR the microparticles are preferably permanently immobilized by covalent binding, sedimentation or magnetic attraction. When using electric magnets, the particles can be in an immobilized or a free floating form during the PCR and are freshly immobilized for each measurement.
1 Emission filter
2 Excitation light filter
3 Camera (CCD or CMOS)
4 LED or laser for excitation
5 Long-distance optical system
6 Stand
7 Z drive
8 Thermocycler
9 X/y drive
10 Detail marking
11 Magnet
12 NucleoSorb or NucleoLink 8-pack
13 Reaction vessel reception in thermoblock
14 Cover
15 Overlay material (e.g. oil)
16 Master mix+sample DNA

### EXAMPLES

### Example 1

### Detection of the Herpes Simplex Virus 1 Pathogen in a Sample

A volume of 5 μl of purified sample DNA from smears of herpes labialis is added to 25 μl of master mix having the following composition:

primer 1 400 nM
primer 2 300 nM
Taq DNA polymerase, nucleotides, magnesium chloride, dissolved in Tris/HCl buffer and Tween 20.

The reaction batch is pipetted into a well of a NucleoLink plate, subsequently transferred into the thermocycler of the measuring device (see FIG. 3) and subjected to thermocyclic amplification. The reaction principle of the method is schematically represented in FIG. 1. The primers 1 and 2 bind to the target sequence of the Herpes simplex virus 1 genome and to the internal standard. Using a DNA polymerase, the fragments flanked by the primers are amplified under suitable conditions. Following denaturing of the double-strand, the probe on the particle surface, which bears a rhodamine label at the 5′ end, can hybridize with the complementary strands of the amplificate. During primer extension in the next reaction cycle, the hybridized probes are degraded by the 5′-3′ exonuclease of the Taq polymerase, and the dye is released, resulting in a decrease of the rhodamine fluorescence as the amount of amplificate increases.

The particles consist of polymethacrylate and have a diameter of 10 μm. The surface of the particles is carboxy-modified, and the 3′-amino-modified capture probes and a 3′-amino-modified and 5′-phosphorylated poly-T(50) linker molecule are coupled to the activated particles according to standard protocols, using carbodiimide coupling. The particles coated with capture probes and linker molecules at a ratio of 1:5 are subsequently bound to the bottom of a NucleoLink plate, using carbodiimide coupling in this case as well.

### Particle 1:

The particle includes e.g. 100 parts of aminocoumarin and e.g. 20 parts of Nile blue (100/30).

### Particle 2:

The particle includes e.g. 100 parts of aminocoumarin and e.g. 100 parts of Nile blue (100/100).

### Particle 3:

The particle includes e.g. 100 parts of aminocoumarin and e.g. 0 parts of Nile blue (100/0).

After pipetting the master mix into the reaction vessels coated with particles and sealing the vessels, the particle fluorescences for aminocoumarin and Nile blue are recorded using a lens with 20-times magnification. The particles of particle populations 1 and 2 are localized and identified. The rhodamine surface fluorescence is preferably recorded between the PCR cycles 20-30, using a lens with 4times magnification in the event of low particle densities and 10-times magnification in the event of high particle densities, until amplificate is unambiguously detected, i.e. the surface fluorescence of the Herpes simplex virus 1-specific particles (particle 1) and/or internal standard particles (particle 2) has clearly decreased compared to the reference fluorescence (particle 3). The reference particles can optionally be used to allow recognition of any non-specific effects such as undesirable nuclease activities or bleaching effects.

### Sequences

Primer 1:

| | |
|---|---|
| 5′-CAT CAC CGA CCC GGA GAG GGA C-3′ | (SEQ ID No: 1) |
| Primer 2: | |
| 5′-GGG CCA GGC GCT TGT TGG TGT A-3′ | (SEQ ID No: 2) |

Herpes simplex virus 1 probe:

(SEQ ID No: 3)

Rhodamine-5′-GGACTTTGTCCTCACCGCCGAACTGATTTTTTTTTTT
TTTT-3′-particle 1

Internal standard probe:

(SEQ ID No: 4)

Rhodamine-5′-GACCGCTTGCTGCAACTCTCTCAGTTTTTTTTTTTTT
T-3′-particle 2

Probe for reference particles to exclude non-specific effects:

(SEQ ID No: 5)

Rhodamine-5′-TTTTTTTTTTTTTTTTTTTTTTTT-3′-
particle 3

Internal standard:

(SEQ ID No: 6)

5′-CATCACCGAC CCGGAGAGGG ACCCAGCGTG GACCGCTTGC
TGCAACTCTC TCAGGGCCAG GCGGTGAAGG GCAATCAGCT
GTTGCCCGTC TCGCTGGTGA AAAGAAAAAC CACCCTGGCG
CCCAATACGC AAACCGCCTA CACCAACAAG CGCCTGGCCC-3′

The multiplex level can be increased by simultaneous amplification of further target sequences, e.g. Herpes simplex virus 2, in the reaction batch and detection thereof on further particle populations using the specific capture probes each time.

This test format can easily be transferred to characterize enzymes such as proteases, phosphatases, glycosidases having the property of degrading fluorescence-labeled acceptor molecules on the surface of the particles.

### Example 2

### Kinase Assay to Detect Different Substrate Specificities

A volume of 5 μl of an ERK1-containing sample is added to 25 μl of a reaction mix having the following composition:

ATP, magnesium chloride, EGTA, DTT, Tris/HCl buffer and Tween 20, optionally enzyme inhibitors, rhodamine-labeled anti-phosphopeptide antibodies of optionally different specificity. As an alternative to the anti-phosphopeptide antibody it is also possible to use phospho group-specific fluorescent dyes. In the event of other modifications such as glycosylations it is also possible to use other modification-specific fluorescent dyes.

To test kinase inhibitors, various kinase inhibitors such as staurosporin are added to different reaction batches in the wells of a microtest plate, and the reaction on phosphorylatable peptides and control peptides immobilized on particles is recorded. When using different phosphorylatable peptides, including control peptides, comparison of different substrate specificities in the multiplex batch is possible as an alternative.

If a peptide can undergo phosphorylation, the rhodamine-labeled anti-phospho group antibody will bind simultaneously to generate a measurable ligand fluorescence.

The particles consist of polymethacrylate and have a diameter of 10 μm. The surface of the particles is carboxy-modified, and the activated particles are coupled by means of carbodiimide coupling according to standard protocols. The particles coated with peptides and optionally with linker molecules at a ratio of 1:5 are subsequently bound to the bottom of a NucleoLink plate, using carbodiimide coupling in this case as well. Alternatively, the particles can be immobilized by means of gravity or magnetic sedimentation.

The following particles are used in accordance with FIG. 2:

### Particle 1:

The core includes e.g. 100 parts of aminocoumarin and e.g. 100 parts of Nile blue (100/100). The central layer includes e.g. 100 parts of fluorescein and the outer layer includes e.g. 50 parts of aminocoumarin and e.g. 100 parts of Nile blue.

### Particle 2:

The core includes e.g. 100 parts of aminocoumarin and e.g. 100 parts of Nile blue (100/100). The central layer includes e.g. 100 parts of fluorescein and the outer layer includes e.g. 100 parts of aminocoumarin and e.g. 100 parts of Nile blue.

Particle 1 bears a peptide phosphorylatable by ERK1, and particle 2 bears a similar but non-phosphorylatable peptide for reference purposes. When using additional microparticle populations, additional phosphorylatable peptides can be integrated in the test so that the multiplex level of the analysis is increased.

After recording the particle fluorescences for aminocoumarin and Nile blue with a 20-times magnification lens and localizing and identifying the particles of particle populations 1 and 2, the reaction mix is pipetted into the reaction vessels coated with particles and the vessels are sealed. The development of rhodamine ligand fluorescence as a result of the kinase reaction is recorded in dependence on the reaction velocity at different points in time, using a lens with 4-times magnification in the event of low particle densities and 10-times magnification in the event of high particle densities, until a clear signal is detected.

Alternatively, many other uses for the detection of antigens and antibodies can be established according to the principle set forth herein, with and without simultaneously proceeding enzyme reaction.

### Legend to the Figures

### →Primer

[symbol] Capture probe with fluorophore 1

### Fluorophore 1

[symbol] Hybridized nucleic acid strands
—Target (amplificate [FIG. 1], peptides [FIG. 2]) [symbol]
Taq polymerase [symbol]
Central layer with magnetic particles and 100 parts of fluorophore, e.g. fluorescein

### Particle

[symbol] Anti-phospho group antibody
P Phospho group

1-37. (canceled)

38. A method for the multiplex analysis of a plurality of analytes, comprising the steps of:
a) using a support which has at least two different microparticle populations immobilized thereon, said different microparticle populations differing in their fluorescence coding and at least two of the differently fluorescence-encoded microparticle populations essentially including microparticles occupied by a particular, specific acceptor molecule population, said acceptor molecule populations of said at least two differently fluorescence-encoded microparticle populations being different from each other;
b) measuring the fluorescence of the support from step a) with an optical resolution 1 prior to contacting the support with the sample to be analyzed,
said resolution 1
permitting differentiation of microparticle singlets, doublets, triplets, multiplets and monolayers, and
allowing determination of the localized position of individual immobilized microparticles of the respective at least two different microparticle populations on the support, taking into account the different fluorescence coding of the at least two different microparticle populations;
c) contacting the support from step a) with the sample to be analyzed, the interaction of the respective analyte with the analyte-specific acceptor molecule on the corresponding immobilized microparticle causing a change in fluorescence;
d) performing at least one additional measurement of the fluorescence of the support during or after contacting or starting the reaction in accordance with step c), using a resolution 2;
e) assigning the fluorescence values measured with resolution 2 to the individual microparticle singlets, doublets, triplets, multiplets and monolayers locally identified on the support in accordance with step b) and assigned to a particular acceptor molecule population;
f) determining the change in fluorescence for each locally identified microparticle singlet and each microparticle in a doublet, triplet, multiplet and monolayer on the support by contacting in accordance with step c).

39. The method according to claim 38, wherein the patterned or non-patterned support in accordance with claim 1, step a), has planar areas allowing microscoping.

40. The method according to claim 38, wherein the microparticles are fluorescence-encoded by means of one or more fluorescent dyes inside the particle or on the particle surface and, in addition, size-encoded via the particle size or structurally encoded through morphological patterns, thereby allowing assignment to distinct microparticle populations.

41. The method according to claim 38, wherein the microparticles are constituted of a core and at least one shell, wherein the materials of core and shell may differ with respect to composition, shape, density, transparency, modifiability and have different fluorescence codings with at least one fluorescent dye, in which context different or identical fluorescent dyes can be used.

42. The method according to claim 38, wherein the support in accordance with claim 38, step a), additionally has living or destroyed cells or cells in combination with microparticles immobilized thereon.

43. The method according to claim 38, wherein the analyte is labeled with a ligand fluorescence or a quencher either directly or indirectly via another molecule.

44. The method according to claim 38, wherein the fluorescence is reduced by quenching of the surface fluorescence or detachment of the surface fluorescence from the particle surface during degradation of the acceptor molecules in the course of the reaction.

45. The method according to claim 38, wherein the increase in ligand fluorescence occurs as a result of terminating the quenching of reporter systems, through FRET or local accumulation of ligand fluorescence molecules on the particle surface via interaction with acceptor molecules or analyte and through displacement of quencher molecules during the reaction.

46. The method according to claim 45, wherein the reaction mixture is added with a quencher dye or light-absorbing nanoparticles so as to increase the contrast of ligand fluorescence.

47. The method according to claim 38, wherein a measuring device for the measurement of changes in fluorescence is used, said device comprising:
a fully automatic control by means of a computer;
a thermocycler for rapid temperature control of the samples, with a heating/cooling rate of 3-20° C. per second, which has a reaction section with a plurality of temperature-controllable receiving means for supports in accordance with claim 38, step a);
a positioning means for the thermocycler and/or the reaction environment, which can be controlled via thermocycles;
one or more illumination means associated with the reaction section, by means of which excitation light can be radiated;
an optical means preferably suitable for incident-light or transmitted-light fluorescence detection using appropriate optical filters;
one or more detector means (e.g. CCD, CMOS) generating images depending on a measured fluorescence intensity; and
an evaluation unit which generates measurement values from the images.

48. The method according to claim 38, wherein during recording additional points of measurement, only the surface or ligand fluorescence of a spatial coordinate-defined pixel number of the particles preselected according to measuring point 1 is recorded and processed further.

49. The method according to claim 38, wherein the different particle fluorescences are related to each other and/or to the surface fluorescence or the particle size in order to decode the at least two different microparticle populations.

50. The method according to claim 38, wherein the different particle fluorescences of different particle layers are related to each other and/or to the surface/ligand fluorescence or the particle size in order to decode the microparticle populations and/or reference the surface or ligand fluorescence.

51. The method according to claim 38, wherein the measurement values of the additional measuring points of a measuring series of one and the same site of measurement recorded over time or in different reaction cycles are referenced to one or more measurement values of the same measuring series, the same site of measurement or other sites of measurement.

52. A kit for use in a method according to claim 38, wherein the kit includes preformulated reagents comprising at least one microparticle population coated with acceptor molecules.
